(19) United States
(12) Patent Application Publication
(10) Pub. No.: US 2026/0120439 A1
(43) Pub. Date: Apr. 30, 2026

(54) LOCALIZATION-AWARE CONFIDENCE CALIBRATION FOR MEDICAL DECISION MAKING

(71) Applicant: NEC Laboratories America, Inc., Princeton, NJ (US)

(72) Inventors: Honglu Zhou, Menlo Park, CA (US); Zachary Izzo, Cranbury, NJ (US); Alexandru Niculescu-Mizil, Plainsboro, NJ (US); Eric Cosatto, Red Bank, NJ (US)

(21) Appl. No.: 18/926,709

(22) Filed: Oct. 25, 2024

Related U.S. Application Data
(60) Provisional application No. 63/595,935, filed on Nov. 3, 2023.
(60) Provisional application No. 63/548,303, filed on Nov. 13, 2023.

(51) Int. Cl. G06V 10/774; G06V 10/25; G06V 10/764; G06V 10/776; G06V 10/94; G06V 20/50; G06V 20/70; G16H 50/20

(52) U.S. Cl. CPC G06V 10/7747; G06V 10/25; G06V 10/764; G06V 10/776; G06V 10/955; G06V 20/50; G06V 20/70; G16H 50/20; G06V 2201/03

(57) ABSTRACT

Methods and systems for model calibration include training an object detection model to generate confidence scores using calibration that is based on confidence, correlation, and matching, with accuracy of a location of bounding boxes being used with accuracy of object labels to keep the confidence scores close to an actual probability of correctness. Object detection is performed on an image using the object detection model to generate a bounding box around an object, a label for the object, and a confidence score. An action is performed responsive to the object and the confidence score.

### RELATED APPLICATION INFORMATION

This application claims priority to U.S. Patent Application No. 63/595,935, filed on Nov. 3, 2023, and to U.S. Patent Application No. 63/548,303, filed Nov. 13, 2023, each incorporated herein by reference in its entirety.

### BACKGROUND

### Technical Field

The present invention relates to object detection and, more particularly, to confidence calibration for object detection.

### Description of the Related Art

Machine learning models can perform a variety of different tasks, including computer vision tasks such as object detection. However, the utility of these models is limited by their accuracy, and further by the ability of the user to measure that accuracy. Some models have the ability to assess a degree of confidence in their own predictions, but such models may be overconfident in their predictions, asserting a high degree of confidence for an output that turns out to be inaccurate. This can be problematic in high-stakes applications, such as healthcare and climate prediction, where the costs of trusting an inaccurate prediction can be high.

### SUMMARY

A method for object detection calibration includes training an object detection model to generate confidence scores using calibration that is based on confidence, correlation, and matching, with accuracy of a location of bounding boxes being used with accuracy of object labels to keep the confidence scores close to an actual probability of correctness. Object detection is performed on an image using the object detection model to generate a bounding box around an object, a label for the object, and a confidence score. An action is performed responsive to the object and the confidence score.

A system for object detection calibration includes a hardware processor and a computer readable storage medium that stores a computer program. When executed by the hardware processor, the computer program causes the hardware processor to train an object detection model to generate confidence scores using calibration that is based on confidence, correlation, and matching, with accuracy of a location of bounding boxes being used with accuracy of object labels to keep the confidence scores close to an actual probability of correctness, to perform object detection on an image using the object detection model to generate a bounding box around an object, a label for the object, and a confidence score, and to perform an action responsive to the object and the confidence score.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram illustrating object detection on a scene, in accordance with an embodiment of the present invention;

FIG. 2 is a block/flow diagram showing the training and use of an object detection model with location-aware calibration, in accordance with an embodiment of the present invention;

FIG. 3 is a diagram illustrating a road scene where calibrated object detection can be used to confidently navigate a hazardous scenario, in accordance with an embodiment of the present invention;

FIG. 4 is a diagram of a self-driving vehicle that can use calibrated object detection to navigate a road scene, in accordance with an embodiment of the present invention;

FIG. 5 is a block diagram of a computing device that can be used to perform location-aware calibration of an object detection model during training, in accordance with an embodiment of the present invention;

FIG. 6 is a diagram of an exemplary neural network architecture that can be used to implement part of an object detection model, in accordance with an embodiment of the present invention;

FIG. 7 is a diagram of an exemplary deep neural network architecture that can be used to implement part of an object detection model, in accordance with an embodiment of the present invention; and

FIG. 8 is a block diagram of a healthcare facility where object detection with location-aware calibration is used to inform medical decision making.

### DETAILED DESCRIPTION OF PREFERRED EMBODIMENTS

Machine learning models may be configured to generate a confidence in their predictions that is calibrated, so that the stated confidence matches a true level of accuracy for the prediction. To that end, the model may be trained with an auxiliary loss function that uses ancillary information from the input to improve the confidence estimation.

For example, in the specific context of an object detection model, training may be performed with a localization-aware loss function, which takes into account not only the confidence of the predicted class of object, but also the spatial accuracy of the bounding box for each object that is detected. By calibrating the model during training, this approach makes a held-out calibration dataset unnecessary, leading to improved data efficiency.

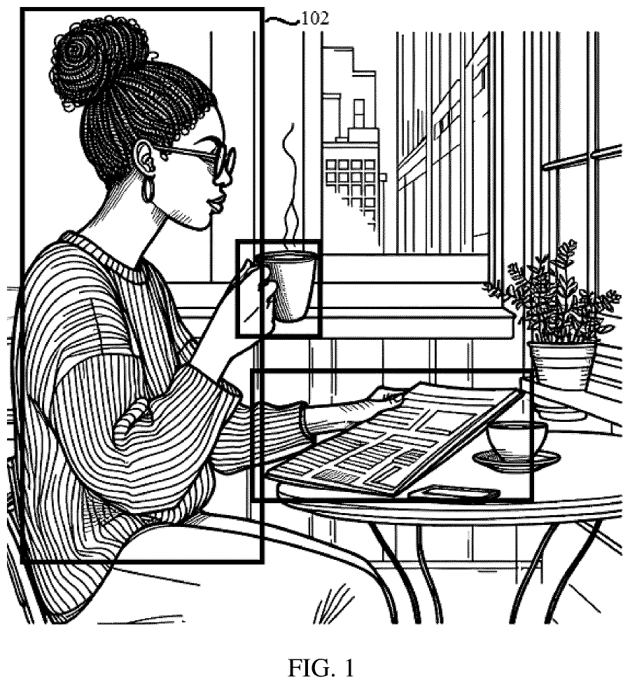

Referring now to FIG. 1, an exemplary image is shown that has had object detection performed on it. In particular, the image shows a number of bounding boxes 102 around different identified objects in the image. The bounding boxes 102 are used to identify the locations of objects and may have accompanying text labels, for example indicating “woman,” “cup,” and “newspaper.”

When an object detection model is used to process an input image, it outputs bounding boxes 102, any associated labels, and a confidence score. The confidence score is a measure of how likely the label is to correctly identify the identified object. For example, the model may have a lower confidence when identifying an object that is blurry within the image, or that is partially occluded. When the object detector is trained to generate these confidence scores, an objective function may be used that encourages model accuracy as well as calibration to ensure that the model is correct about that accuracy.

For example, a calibration loss may be used during training to quantify the calibration according to an evaluation metric. This encourages higher confidence for accurate object labels and lower confidence for inaccurate ones, which improves the reliability and trustworthiness of the object detection model. The loss function may thus consider localization and classification jointly to eliminate inconsistencies in the training signal, thus providing a better calibrated model while preserving accuracy. Object localization may be implemented using precise intersection over union (IoU) scores. This IoU-sensitive approach makes the auxiliary loss suitable for use as a calibration loss.

A well-calibrated object detection model is one in which its confidence of prediction aligns with the actual probability of correctness. A dataset

$$𝒟 = {〈 ( {x}_{i} , {y}_{i}^{*} ) 〉}_{i = 1}^{N}$$

includes N pairs of images xᵢ and their corresponding ground-truth labels

$${y}_{i}^{*}$$

drawn from a joint distribution [symbol] (χ, [symbol] ). Here xᵢ ∈ [symbol] denotes an image and

$${y}_{i}^{*} ∈ 𝒴 = 1 , … , K$$

denotes the associated ground-truth class label. The total number of classes is K, while H, W, and C refer to the height, width, and number of channels of an image, respectively.

A classification model, denoted as [symbol] _{cls}, predicts a class label ŷ along with a confidence score ŝ, with perfect calibration being defined as:

$$ℙ ( \hat{y} = {y}^{*} \vert \hat{s} = s ) = s , ∀ s ∈ [ 0 , 1 ]$$

where [symbol] (ŷ=y*|ŝ=s) is the accuracy corresponding to a certain confidence score s. For the model to be considered well-calibrated, this accuracy should precisely match with the predicted confidence.

For object detection, datasets come with annotations that specify the location and category of each object within the images. The bounding box annotation for an object is b*∈ [symbol] =[0,1]⁴ and its associated class label is y*. An object detection model [symbol] _{det} that predicts an object's location as \hat{b} and its class label ŷ and generates a class confidence score ŝ. This prediction is deemed a true positive if it satisfies two conditions: First, the IoU between \hat{b} and b* is not lower than a predefined threshold γ and, second, ŷ is the same as y*. Mathematically, this criterion can be expressed as [symbol] [IoU(\hat{b}, b*)≥γ] [symbol] [ŷ=y*], where [symbol] is an indicator function that takes the value 1 when the condition in the brackets is true and takes the value 0 otherwise. Then, for object detection, a perfect calibration can be expressed as:

$$ℙ ( U = 1 \vert \hat{s} = s ) = s , ∀ s ∈ [ 0 , 1 ]$$

where U=1 denotes an accurate detection.

Expected calibration error (ECE) is a widely used metric to quantify the miscalibration of a classification model. It measures the expected deviation of accuracy from the confidence for all confidence levels:

$${\hat{s}}_{} [ \vert ℙ ( \hat{y} = {y}^{*} \vert \hat{s} = s ) - s \vert ]$$

The ECE metric approximates this by dividing the continuous confidence space of § into M equally spaced bins:

$$ECE = {\sum }_{m = 1}^{M} \frac{\vert B ( m ) \vert}{\vert 𝒟 \vert} \vert acc ( m ) - conf ( m ) \vert$$

where B(m) denotes the set of samples in m^{th} bin, and | [symbol] | is the total number of samples. acc(m) and conf(m) denote the average accuracy and average confidence of predictions in the m^{th} bin, respectively.

Similar to ECE for classification, detection ECE (D-ECE) may be the expected deviation of precision from the confidence for all confidence levels:

$${\hat{s}}_{} [ \vert ℙ ( U = 1 \vert \hat{s} = s ) - s \vert ]$$

D-ECE approximates this by dividing the continuous confidence space into M equally spaced bins:

$$D - ECE = {\sum }_{m = 1}^{M} \frac{\vert B ( m ) \vert}{\vert 𝒟 \vert} \vert pre c ( m ) - conf ( m ) \vert$$

where B(m) denotes the set of object instances in m^{th} bin, and | [symbol] | is the total number of object instances. Unlike classification, for object detection, precision is used instead of average accuracy. Specifically, precision is the ratio of the number of true positives to the total number of positive detections. prec(m) and conf(m) denote the precision and average confidence of detections in m^{th} bin, respectively.

As D-ECE does not account for the fact that two object detectors, while having the same precision, might differ significantly in terms of localization quality. Therefore, Localization Aware ECE (LA-ECE) is defined by approximating D-ECE as:

$$LA - ECE = {\sum }_{k = 1}^{K} {\sum }_{m = 1}^{M} \frac{\vert {B}^{k} ( m ) \vert}{\vert {\hat{𝒟}}^{k} \vert} \vert pre {c}^{k} ( m ) \cdot {IoU}^{k} ( m ) - {conf}^{k} ( m ) \vert$$

where [symbol] is the set of object detections with class label k, and \hat{B}^{k}(m) denotes the set of object detections with class label k in m^{th} bin. prec ^{k}(m) denotes the precision of the m^{th} bin for class label k, and IoU^{k}(m) is the average IoU of true positive detections with class label k in m^{th} bin. Furthermore, conf^{k}(m) denote the average confidence of detections with class label k in m^{th} bin. Unlike D-ECE, LA-ECE additionally considers the average IoU score of true positive detections in m^{th} bin in the measurement for detection correctness.

The tendency of Deep Neural Networks (DNNs) to exhibit miscalibration stems from an absence of explicit supervision guiding the model to associate higher confidence levels with precise predictions and lower confidence levels with imprecise predictions. In light of this, an auxiliary loss function may be used to calibrate DNN-based object detectors, establishing a train-time method for enhanced calibration for object detection.

Motivated by the way miscalibration can be quantified through calculating the deviation between the measures of prediction correctness and prediction confidence, localization-aware matching of correctness and confidence (LoMaCC) may be used as an auxiliary loss during the training phase. The correctness of a detection i is determined as:

$${q}_{i} \cdot {b}_{i} ( {γ}_{t} )$$

where qᵢ∈{0,1} and bᵢ(γₜ)∈{0,1}. The term qᵢ=1 when

$${\hat{y}}_{i} = {y}_{i}^{*} ,$$

i.e., the predicted class is the same as the ground-truth class, otherwise qᵢ=0.bᵢ(γₜ)=1 when IoU(\hat{b}ᵢ,

$${b}_{i}^{*} ) \geq {γ}_{t} ,$$

i.e., the IoU between box i and its ground-truth box is not lower than γₜ; otherwise bᵢ(γₜ)=0. In other words, the terms qᵢ and bᵢ also operate as indicator functions. This correctness establishes that a correct detection satisfies two conditions: accurate classification and precise localization. Normally, during the early phases of neural network training, the predicted bounding boxes exhibit low IoU scores. Consequently, if γₜ is set to a high constant value such as 0.7, the values of bᵢ(γₜ) are predominantly zeros. In order to better accommodate to different phases of the training process, γₜ is a dynamic IoU threshold whose value depends on the current training epoch. Specifically:

$${γ}_{t} = min ( {γ}_{max} , {γ}_{max} \cdot \frac{t}{r \cdot T} )$$

where t and T represent the current epoch and the predetermined total number of training epochs, respectively, while r dictates the transition point to the maximal IoU threshold value γₘₐₓ. Empirically, the threshold scheduler hyper-parameter r may be set to an exemplary value of 35%, and γₘₐₓ to 0.7. When t>r·T—once the training phase surpasses 35% of the overall progression of training—γₜ invariably adopts the value of γₘₐₓ; otherwise its value scales linearly with training progress until it reaches γₘₐₓ.

Confidence of a detection i can be calculated as ŝᵢ, the confidence of the predicted class, where \hat{b}ᵢ and

$${b}_{i}^{*}$$

denote the predicted and the ground-truth bounding boxes. This is a localization-aware confidence measurement for predictions in the context of object detection. This is due to the consideration of the exact values of the IoU scores.

The first component in LoMaCC quantifies the averaged absolute difference between the correctness and confidence over a minibatch of samples:

$${ℒ}_{match} = \vert \frac{1}{{N}_{b}} {\sum }_{i = 1}^{{N}_{b}} {q}_{i} \cdot {b}_{i} ( {γ}_{t} ) - \frac{1}{{N}_{b}} {\sum }_{i = 1}^{{N}_{b}} {\hat{s}}_{i} \vert$$

where N_{b} represent the size of the minibatch. The detections of true positives and false positives are categorized into four non-overlapping groups: (1) correct-and-confident, (2) incorrect-and-not-confident, (3) incorrect-and-confident, and (4) correct-and-not-confident detections.

For the correct-and-confident category, detections are both accurate and made with high confidence. In the most ideal scenario, the expected value of [symbol] _{match} for correct-and-confident detections would be

$$\frac{1}{{N}_{b}} {\sum }_{i = 1}^{{N}_{b}} \vert 1 \cdot 1 - 1 \cdot 1 \vert ,$$

which simplifies to 0. Conversely, in the incorrect-and-not-confident category, detections are inaccurate and made with low confidence. This can be due to errors in either classification or localization, paired with a lack of confidence in either the classification or localization. In extreme cases, this combination yields a [symbol] _{match} value of 0. Similarly, for the incorrect-and-confident and correct-and-not-confident categories, the expected extreme loss value of [symbol] _{match} is 1, indicating a notable discrepancy between the correctness and the confidence in the result. The value of [symbol] _{match} is in the range between 0 and 1. Therefore, striving to minimize [symbol] _{match} promotes more incorrect-and-confident and incorrect-and-not-confident detections, and fewer incorrect-and-confident and correct-and-not-confident detections.

Nevertheless, minimizing the [symbol] _{match} objective can inadvertently result in correct-and-confident detections transitioning into incorrect-and-not-confident detections, while maintaining or even increasing the combined number of correct-and-confident and incorrect-and-not-confident detections. To mitigate this, an additional loss term is used to encourage more correct detections and more confident detections:

$${ℒ}_{c c} = - \frac{1}{{N}_{b}} {\sum }_{i = 1}^{{N}_{b}} {q}_{i} \cdot {b}_{i} ( {γ}_{t} ) - \frac{1}{{N}_{b}} {\sum }_{i = 1}^{{N}_{b}} {\hat{s}}_{i}$$

Furthermore, to avoid the potential undesired behavior that correct-and-not-confident detections may transition into incorrect-and-not-confident detections, [symbol] _{corr} may be used, which corresponds to negative empirical Pearson correlation coefficient:

$${ℒ}_{p e a r s o n} = - \frac{{\sum }_{i = 1}^{{N}_{b}} ( {s}_{i} - \overline{\hat{s}} ) ( {q}_{i} \cdot {b}_{i} ( {γ}_{t} ) - \overline{q \cdot b ( {γ}_{t} )} )}{\sqrt{{\sum }_{i = 1}^{{N}_{b}} {( {s}_{i} - \overline{\hat{s}} )}^{2} \cdot {\sum }_{i = 1}^{{N}_{b}} {( {q}_{i} \cdot {b}_{i} ( {γ}_{t} ) - \overline{q \cdot b ( γ t )} )}^{2}}}$$

where

$$\overline{\hat{s}} = \frac{1}{{N}_{b}} {\sum }_{i = 1}^{{N}_{b}} {\hat{s}}_{i} and \overline{q \cdot b ( {γ}_{t} )} = \frac{1}{{N}_{b}} {\sum }_{i = 1}^{{N}_{b}} {q}_{i} \cdot {b}_{i} ( {γ}_{t} ) . {ℒ}_{p e a r s o n}$$

ensures a population-level linear alignment between correct detections and confident detections.

The train-time auxiliary loss for object detection calibration is computed as:

$${ℒ}_{L o M a C C} = {ℒ}_{m a t c h} + {αℒ}_{c c} + {βℒ}_{p e a r s o n}$$

where α and β are weighting parameters. This loss may be combined with an object detection loss when training an object detection model to jointly train the model for object detection and for calibrated confidence estimation. Minimizing [symbol] _{LOMaCC} implicitly encourages accurate detections to be more confident and inaccurate ones to be less confident, fostering a more reliable and trustworthy object detection system. The localization-aware nature of [symbol] _{LoMaCC} is achieved through the direct utilization of precise IoU scores. Additionally, the IoU threshold utilized in the loss computation dynamically evolves in accordance with the progression of the training process. This IoU-sensitive property and dynamic adjustment of IoU threshold render [symbol] _{LoMaCC} an apt choice as a calibration loss for object detectors.

The auxiliary loss function promotes calibration during training. To show this, the following definitions are applied:

$${ℒ}_{match , i} = {\hat{s}}_{i} \cdot Io {U}_{i} - {q}_{i} \cdot {b}_{i} ( {γ}_{t} ) ,$$

$${ℒ}_{cc , i} = {-}_{i} \cdot {IoU}_{i}$$

$${( Δ \hat{s} IoU )}_{i} = {\hat{s}}_{i} IoU - \overline{\hat{s} IoU} , Δ \hat{s} IoU ∈$$

$${( Δ qb )}_{i} = {q}_{i} {b}_{i} ( {γ}_{t} ) - \overline{qb} , Δ qb ∈$$

Direct computation then yields the follow expressions for the gradients of our proposed loss terms:

$$∇ \vert {ℒ}_{m a t c h , i} \vert = sign ( {ℒ}_{m a t c h , i} ) ( {IoU}_{i} ∇ {\hat{s}}_{i} + {\hat{s}}_{i} ∇ I o {U}_{i} ) ,$$

$$∇ {ℒ}_{c c , i} = - ( {IoU}_{i} ∇ {\hat{s}}_{i} + {\hat{s}}_{i} ∇ {IoU}_{i} ) ,$$

$$∇ {ℒ}_{p e a r s o n} ∝ {{\sum }^{{N}_{b}}}_{i = 1} ( - ‖ Δ \hat{s} IoU ‖ ‖ Δ qb ‖ ( {q}_{i} {b}_{i} ( {γ}_{t} ) - \overline{qb} ) + \frac{( Δ \hat{sIoU} \cdot Δ qb ) ‖ Δ qb ‖}{‖ Δ \hat{sIoU} ‖} ( {\hat{s}}_{i} {IoU}_{i} - \overline{\hat{s} IoU} ) ) \times ( {IoU}_{i} ∇ {\hat{s}}_{i} + {\hat{s}}_{i} ∇ {IoU}_{i} )$$

where v∂w for two vectors v and w means that v=cw for some positive scalar c (i.e., the two vectors point in the same direction). Note that since qᵢ, bᵢ(γₜ)∈{0,1} and ŝᵢ, IoUᵢ∈[0,1], we have [symbol] _{match,i}<0 iff qᵢ=bᵢ(γₜ)=1 (i.e., the model predicts correctly with an accurate bounding box). Otherwise [symbol] _{match,i}>0, in which case either qᵢ=0 (the prediction is incorrect), bᵢ(γₜ)=0 (the bounding box is not accurate), or both. Thus, [symbol] _{match} will encourage the model to increase its confidence and IoU value on correctly predicted samples, and decrease its confidence and IoU value on samples where either the bounding box or prediction is incorrect.

[symbol] _{cc} will always encourage the model to increase its max confidence and IoU values for every sample. However, the contribution to the overall gradient is smaller (by a factor of α) than the contribution from [symbol] _{match}. This is not necessarily encouraging it to increase its confidence for the correct class, just the confidence of the currently predicted class.

To analyze [symbol] ₚₑₐᵣₛₒₙ, each term in the sum for ∇ [symbol] ₚₑₐᵣₛₒₙ is divided into two parts. The first part is

$$- ‖ Δ \hat{s} IOU ‖ ‖ Δ qb ‖ ( {q}_{i} {b}_{i} ( {γ}_{t} ) - \overline{qb} ) ( {IoU}_{i} ∇ {\hat{s}}_{i} + {\hat{s}}_{i} ∇ {IoU}_{i} )$$

Note that qᵢbᵢ(γₜ)−q̅b̅>0 if, and only if, the model makes the correct prediction (both the correct class and bounding box) on the i-th example. In this case, a step of gradient descent will increase the model's confidence and IoU on the i-th sample. Otherwise, if the prediction is incorrect (either due to a bad class prediction or localization), a step of gradient descent will decrease the model's confidence and IoU on the i-th example.

The second part of the gradient is

$$\frac{( Δ \hat{s} IoU \cdot Δ qb ) ‖ Δ qb ‖}{‖ Δ \hat{s} IoU ‖} ( {\hat{s}}_{i} {IoU}_{i} - \overline{\hat{s} IoU} ) ( {IoU}_{i} ∇ {\hat{s}}_{i} + {\hat{s}}_{i} ∇ {IoU}_{i} )$$

For simplicity, it may be assumed that ΔŝIoU·Δqb≥0. This essentially means that the model confidence and IoU values are reasonably well-aligned with the model predictions and thresholded IoU values, which be the case as the model training progresses. In this case, a gradient descent step will decrease model confidence and IoU on the i-th example when its joint confidence/IoU is greater than the average over all samples; otherwise, it will increase the model confidence and IoU on samples where the joint model confidence/IoU is below the average. The net effect is to shrink all model confidence/IoU values towards the mean for all samples. This regularization effect should reduce over and underconfident predictions and improve calibration.

For the version where the IoU calculation is detached when computing ∇ [symbol] _{LOMaCC}, all of the above expressions apply with ∇IoUᵢ=0. Thus, only ŝᵢ will be updated in the qualitative ways described above.

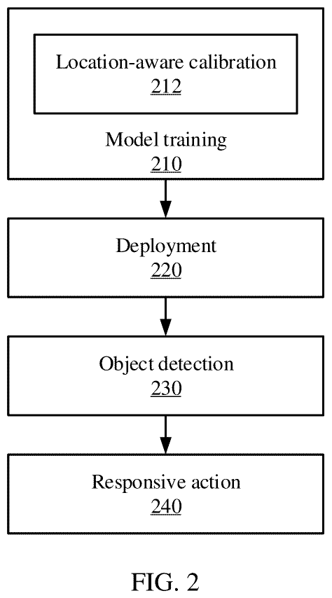

Referring now to FIG. 2, a method of training and using an object detection model with localization-aware calibration is shown. Block 210 performs training using a dataset of training examples, which may include a set of images with object bounding boxes and associated labels. This training includes location-aware calibration, for example by using the [symbol] _{LoMaCC} loss function described above. The resulting trained object detection model accepts unlabeled images as inputs and outputs one or more bounding boxes that correspond to respective objects within the image. Each bounding box may have one or more labels associated with it that describe the identified object, as well as a confidence value that describes how likely the label and bounding box are to be accurate for a given object.

Block 220 deploys the trained model. This deployment may include copying the model parameters to a new environment, such as in the controller of a self-driving vehicle. In other embodiments, the deployment may include enabling an object detection model in a healthcare context, where it can help to diagnose disease and aid in medical decision making. Block 230 then uses the trained model to perform object detection. Based on the output of the trained object detection model, block 240 performs a responsive action.

An exemplary application for the trained model is in self-driving vehicles, where object detection can be used to identify and avoid hazards. The consequences for misidentifying an object can be drastic, and so the improved confidence scores improve a self-driving system's ability to safely and efficiently reach its destination.

For example, in a self-driving application, the object detection 230 may identify one or more objects within a driving scene that have implications for the vehicle's safe operation. An example may include identifying a road hazard, such as an obstruction or pothole, or a pedestrian or another vehicle that is in the vehicle's path. To respond to this object, block 240 may perform a driving action, such as a steering action, a braking action, and/or an acceleration action, to change the vehicle's direction and speed to avoid the hazard.

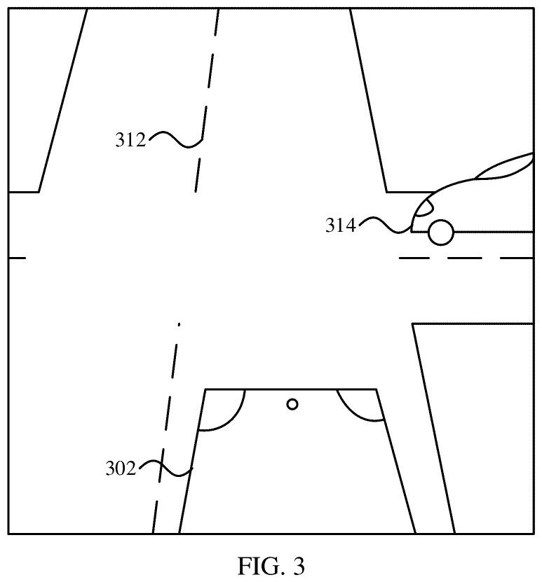

Referring now to FIG. 3, an example road scene is shown. The scene may be captured by a camera that is mounted on a vehicle 302, and may show the surroundings of the vehicle 302 from a particular perspective. It should be understood that multiple such images may be used to show various perspectives, to ensure awareness of the vehicle's entire surroundings. In some cases, a panoramic or 360° camera may be used.

The object detection model may process an image of the scene and identify different objects that are shown in the scene. A controller of the vehicle 302 can then generate an action for how the vehicle 302 should act to reach its destination safely. The model may detect environmental features, such as the road boundary and lane markings 312, as well as moving objects, such as other vehicles 314. Using this information, a navigation or self-driving system in the vehicle 302 can safely navigate through the scene.

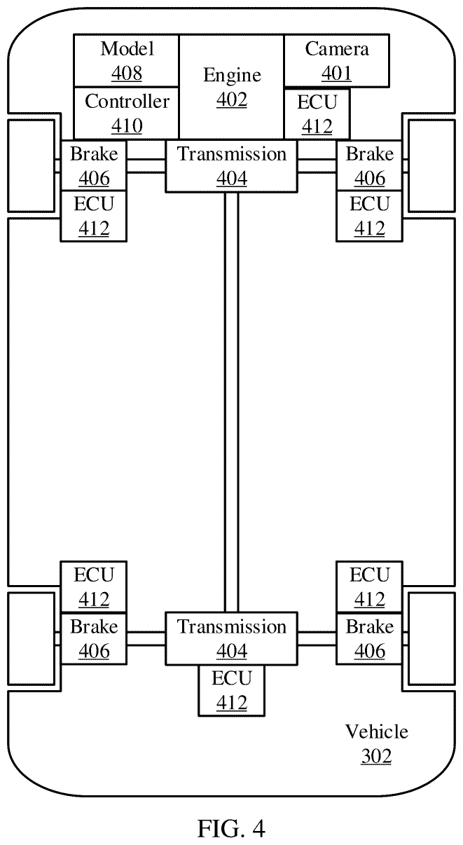

Referring now to FIG. 4, additional detail on a vehicle 302 is shown. A number of different sub-systems of the vehicle 302 are shown, including an engine 402, a transmission 404, and brakes 406. It should be understood that these sub-systems are provided for the sake of illustration, and should not be interpreted as limiting. Additional sub-systems may include user-facing systems, such as climate control, user interface, steering control, and braking control. Additional sub-systems may include systems that the user does not directly interact with, such as tire pressure monitoring, location sensing, collision detection and avoidance, and self-driving.

Each sub-system is controlled by one or more equipment control units (ECUs) 412, which perform measurements of the state of the respective sub-system. For example, ECUs 412 relating to the brakes 406 may control an amount of pressure that is applied by the brakes 406. An ECU 412 associated with the wheels may further control the direction of the wheels. The information that is gathered by the ECUs 412 is supplied to the controller 410. A camera 401 or other sensor (e.g., LiDAR or RADAR) can be used to collect information about the surrounding road scene, and such information may also be supplied to the controller 410.

Communications between ECUs 412 and the sub-systems of the vehicle 302 may be conveyed by any appropriate wired or wireless communications medium and protocol. For example, a car area network (CAN) may be used for communication. The time series information may be communicated from the ECUs 412 to the controller 410, and instructions from the controller 410 may be communicated to the respective sub-systems of the vehicle 302.

The controller 410 uses the output of the object detection model 408, based on information collected from cameras 401, to identify objects and hazards within the scene. The model 408 may, for example, determine a driving action to perform responsive to the present state of the scene. Because the model 408 has been trained on diverse simulated inputs, it will determine a safe and efficient path to its destination.

The controller 410 may communicate internally to the sub-systems of the vehicle 302 and the ECUs 412. Based on detected road fault information, the controller 410 may communicate instructions to the ECUs 412 to avoid a hazardous road condition. For example, the controller 410 may automatically trigger the brakes 406 to slow down the vehicle 302 and may furthermore provide steering information to the wheels to cause the vehicle 302 to move around a hazard.

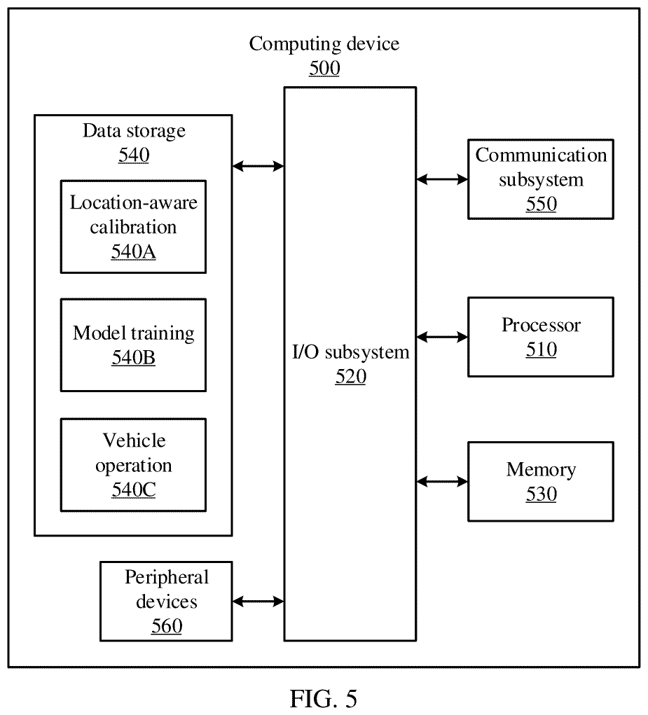

Referring now to FIG. 5, an exemplary computing device 500 is shown, in accordance with an embodiment of the present invention. The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for location-aware calibration, 540B for training planner model, and/or 540C for performing vehicle operation actions using the trained planner model. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

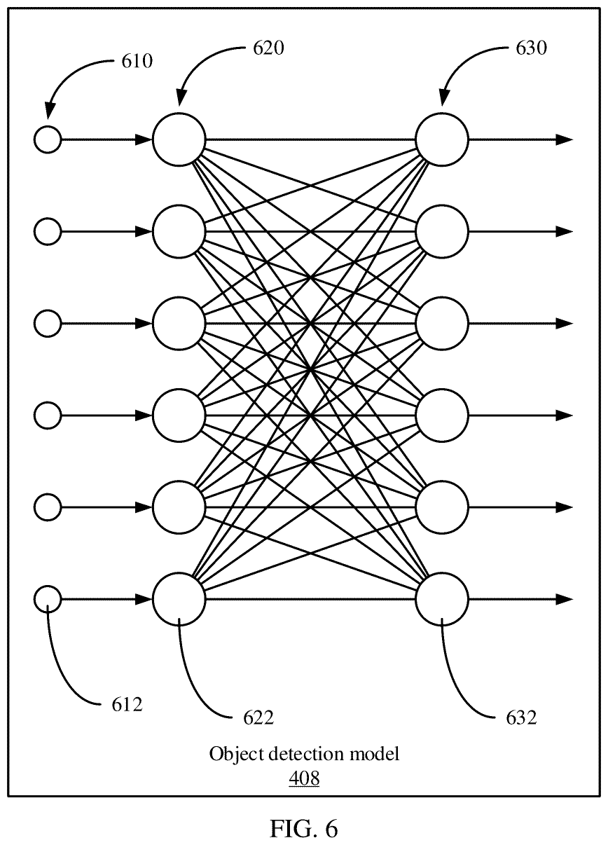

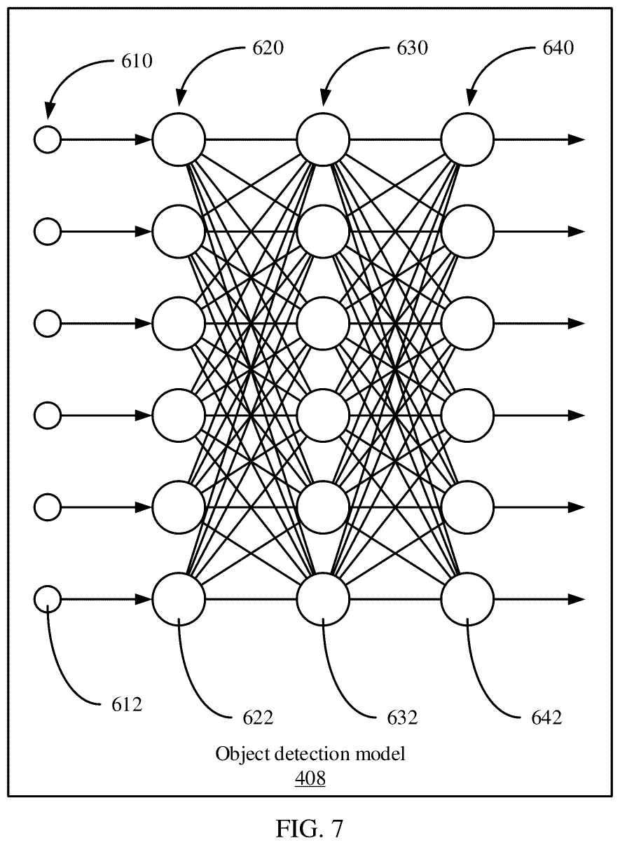

Referring now to FIGS. 6 and 7, exemplary neural network architectures are shown, which may be used to implement parts of the present models, such as the object detection model 408. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

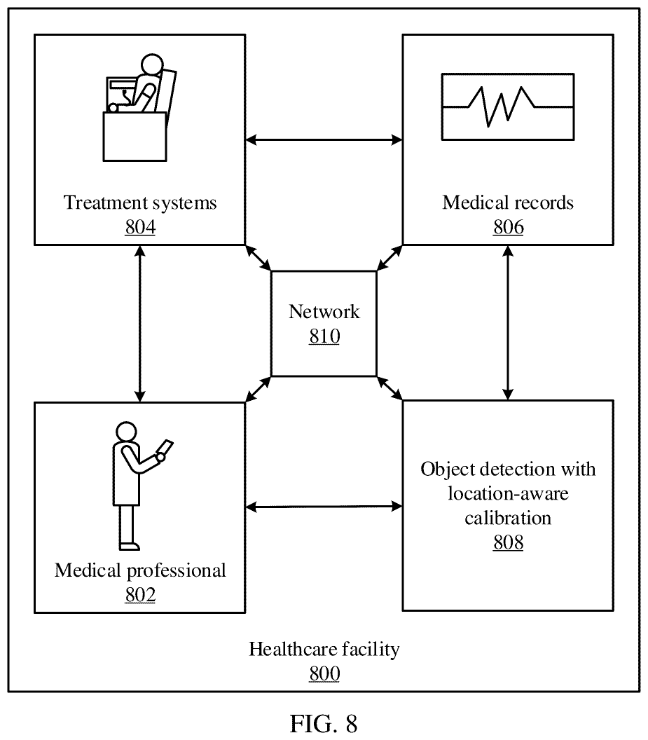

Referring now to FIG. 8, a diagram of information extraction is shown in the context of a healthcare facility 800. Object detection with location-aware calibration 808 may be used to process medical imaging information, such as x-ray images, magnetic resonance imaging (MRI) information, and computed tomography scans stored as medical records 806. The object detection with location-aware calibration 808 can identify medically significant information within such images, such as the presence or absence or tumors, their locations, and their sizes.

The healthcare facility may include one or more medical professionals 802 who review information extracted from a patient's medical records 806 to determine their healthcare and treatment needs. These medical records 806 may include self-reported information from the patient, test results, and notes by healthcare personnel made to the patient's file. Treatment systems 804 may furthermore monitor patient status to generate medical records 806 and may be designed to automatically administer and adjust treatments as needed.

Based on information provided by the object detection with location-aware calibration 808, the medical professionals 802 may make medical decisions about patient healthcare suited to the patient's needs. For example, the medical professionals 802 may make a diagnosis of the patient's health condition and may prescribe particular medications, surgeries, and/or therapies.

The different elements of the healthcare facility 800 may communicate with one another via a network 810, for example using any appropriate wired or wireless communications protocol and medium. Thus the object detection with location-aware calibration 808 can receive a query from medical professionals 802 relating to a condition and may formulate a response based on information gleaned from stored medical records 806. The object detection with location-aware calibration 808 may coordinate with treatment systems 804 in some cases to automatically administer or alter a treatment. For example, if the object detection with location-aware calibration 808 indicates a particular disease or condition, then the treatment systems 804 may automatically halt the administration of the treatment.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for model calibration, comprising:
training an object detection model to generate confidence scores using calibration that is based on confidence, correlation, and matching, with accuracy of a location of bounding boxes being used with accuracy of object labels to keep the confidence scores close to an actual probability of correctness;
performing object detection on an image using the object detection model to generate a bounding box around an object, a label for the object, and a confidence score; and
performing an action responsive to the object and the confidence score.

2. The method of claim 1, wherein the training uses a loss function for location-aware matching of correctness and confidence:
$${ℒ}_{LoMaCC} = {ℒ}_{match} + {αℒ}_{c c} + {βℒ}_{pearson}$$
where α and β are weighting parameters, [symbol] _{match} quantifies an averaged absolute difference between the correctness and confidence, [symbol] _{cc} encourages more correct detections and more confident detections, and [symbol] ₚₑₐᵣₛₒₙ is a correlation loss.

3. The method of claim 2, wherein [symbol] _{match} is expressed as:
$${ℒ}_{match} = \vert \frac{1}{{N}_{b}} {\sum }_{i = 1}^{{N}_{b}} {q}_{i} \cdot {b}_{i} ( {γ}_{t} ) - \frac{1}{{N}_{b}} {\sum }_{i = 1}^{{N}_{b}} {\hat{s}}_{i} \vert$$
where N_{b} is a minibatch size, qᵢ indicates whether a predicted class is the same as a ground-truth class, bᵢ(γₜ) indicates whether a predicted bounding box and a ground-truth bounding box differ by more than a threshold γₜ, and ŝᵢ is a confidence.

4. The method of claim 3, wherein the threshold γₜ dynamically evolves through the training.

5. The method of claim 4, wherein the threshold is defined as:
$${γ}_{t} = min ( {γ}_{max} , {γ}_{max} \cdot \frac{t}{r \cdot T} )$$
where t and T represent a current epoch and a predetermined total number of training epochs and r is a threshold scheduler hyper-parameter.

6. The method of claim 3, wherein bᵢ(·) uses an intersection over union (IoU) score between the predicted bounding box and the ground-truth bounding box.

7. The method of claim 2, wherein [symbol] _{cc} is expressed as:
$${L}_{c c} = - \frac{1}{{N}_{b}} {\sum }_{i = 1}^{{N}_{b}} {q}_{i} \cdot {b}_{i} ( {γ}_{t} ) - \frac{1}{{N}_{b}} {\sum }_{i = 1}^{{N}_{b}} {\hat{s}}_{i}$$
where N_{b} is a minibatch size, qᵢ indicates whether a predicted class is the same as a ground-truth class, bᵢ(γₜ) indicates whether a predicted bounding box and a ground-truth bounding box differ by more than a threshold γₜ, and ŝᵢ is a confidence.

8. The method of claim 2, wherein the training combines the loss function for location-aware matching of correctness and confidence with a loss function for object detection to jointly train the object detection with calibration.

9. The method of claim 1, wherein the object detection model is a machine learning model that is trained using medical image information and the label is used for medical decision making.

10. The method of claim 9, wherein action includes automatically altering a patient's treatment responsive to the label and the confidence score.

11. A system for model calibration, comprising:
a hardware processor; and
a computer readable storage medium that stores a computer program which, when executed by the hardware processor, causes the hardware processor to:
train an object detection model to generate confidence scores using calibration that is based on confidence, correlation, and matching, with accuracy of a location of bounding boxes being used with accuracy of object labels to keep the confidence scores close to an actual probability of correctness;
perform object detection on an image using the object detection model to generate a bounding box around an object, a label for the object, and a confidence score; and
perform an action responsive to the object and the confidence score.

12. The system of claim 11, wherein the training uses a loss function for location-aware matching of correctness and confidence:
$${ℒ}_{LoMaCC} = {ℒ}_{match} + {αℒ}_{c c} + {βℒ}_{pearson}$$
where α and β are weighting parameters, [symbol] _{match} quantifies an averaged absolute difference between the correctness and confidence, [symbol] _{cc} encourages more correct detections and more confident detections, and [symbol] ₚₑₐᵣₛₒₙ is a correlation loss.

13. The system of claim 12, wherein [symbol] _{match} is expressed as:
$${ℒ}_{match} = \vert \frac{1}{{N}_{b}} {\sum }_{i = 1}^{{N}_{b}} {q}_{i} \cdot {b}_{i} ( {γ}_{t} ) - \frac{1}{{N}_{b}} {\sum }_{i = 1}^{{N}_{b}} {\hat{s}}_{i} \vert$$
where N_{b} is a minibatch size, qᵢ indicates whether a predicted class is the same as a ground-truth class, bᵢ(γₜ) indicates whether a predicted bounding box and a ground-truth bounding box differ by more than a threshold γₜ, and ŝᵢ is a confidence.

14. The system of claim 13, wherein the threshold γₜ dynamically evolves through the training.

15. The system of claim 14, wherein the threshold is defined as:
$${γ}_{t} = min ( {γ}_{max} , {γ}_{max} \cdot \frac{t}{r \cdot T} )$$
where t and T represent a current epoch and a predetermined total number of training epochs and r is a threshold scheduler hyper-parameter.

16. The system of claim 13, wherein bᵢ(·) uses an intersection over union (IoU) score between the predicted bounding box and the ground-truth bounding box.

17. The system of claim 12, wherein [symbol] _{cc} is expressed as:
$${L}_{c c} = - \frac{1}{{N}_{b}} {\sum }_{i = 1}^{{N}_{b}} {q}_{i} \cdot {b}_{i} ( {γ}_{t} ) - \frac{1}{{N}_{b}} {\sum }_{i = 1}^{{N}_{b}} {\hat{s}}_{i}$$
where Ng is a minibatch size, q; indicates whether a predicted class is the same as a ground-truth class, bᵢ(γₜ) indicates whether a predicted bounding box and a ground-truth bounding box differ by more than a threshold γₜ, and ŝᵢ is a confidence.

18. The system of claim 12, wherein the training combines the loss function for location-aware matching of correctness and confidence with a loss function for object detection to jointly train the object detection with calibration.

19. The system of claim 11, wherein the object detection model is a machine learning model that is trained using medical image information and the label is used for medical decision making.

20. The system of claim 19, wherein action includes automatic alteration of a patient's treatment responsive to the label and the confidence score.
